(19) United States
(12) Patent Application Publication
(10) Pub. No.: US 2006/0089711 A1
(43) Pub. Date: Apr. 27, 2006

(54) Multifilament anchor for reducing a compass of a lumen or structure in mammalian body

(75) Inventor: Mark J. Dolan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/233,592

(22) Filed: Sep. 22, 2005

Related U.S. Application Data
(60) Provisional application No. 60/622,359, filed on Oct. 27, 2004.

(51) Int. Cl. A61F 2/24; A61B 17/04; A61B 17/12

(52) U.S. Cl. 623/2.37; 606/232; 606/157; 606/151

(57) ABSTRACT

A system for reducing a compass of an opening or structure in a mammalian body comprises an anchor having a central aperture, a tensioner having a plurality of openings, and a plurality of filaments, each including a retaining member affixed to a distal portion of the filament. The tensioner is receivable within the anchor central aperture. A proximal portion of each filament is receivable within a tensioner opening. A method of reducing a compass of a lumen or structure in a mammalian body comprises delivering the anchor to a first location proximate target tissue, delivering the filaments to a second location proximate the target tissue, threading the filaments through the anchor and the tensioner openings, positioning the tensioner in the anchor aperture, retaining the filaments in the tensioner, and rotating the tensioner to twist the filaments, thereby shortening the length of the filaments and increasing the tension across the system.

### RELATED APPLICATIONS

This application claims the benefit of U.S. Provisional Patent Application 60/622,359 filed Oct. 27, 2004.

### TECHNICAL FIELD

This invention relates generally to medical devices and particularly to a device, system, and method for reducing a compass of a lumen or structure in a mammalian body.

### BACKGROUND OF THE INVENTION

The heart is a four-chambered pump that moves blood efficiently through the vascular system. Blood enters the heart through the vena cava and flows into the right atrium. From the right atrium, blood flows through the tricuspid valve and into the right ventricle, which then contracts and forces blood through the pulmonic valve and into the lungs. Oxygenated blood returns from the lungs and enters the heart through the left atrium and passes through the bicuspid mitral valve into the left ventricle. The left ventricle contracts and pumps blood through the aortic valve into the aorta and to the vascular system.

The mitral valve consists of two leaflets (anterior and posterior) attached to a fibrous ring or annulus. In a healthy heart, the mitral valve leaflets overlap during contraction of the left ventricle and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

It is common medical practice to treat mitral valve regurgitation by valve replacement or repair. Valve replacement involves an open-heart surgical procedure in which the patient's mitral valve is removed and replaced with an artificial valve. This is a complex, invasive surgical procedure with the potential for many complications and a long recovery period.

Mitral valve repair includes a variety of procedures to reshape or reposition the leaflets to improve closure of the valve during ventricular contraction. Correction of the regurgitation may not require repair of the valve leaflets themselves, but simply a reduction in the size of a distended mitral valve annulus. A common repair procedure involves implanting an annuloplasty ring on the mitral valve annulus. The annuloplasty ring generally has a smaller diameter than the distended annulus, and when sutured to the annulus, the annuloplasty ring draws the annulus into a smaller configuration, bringing the mitral valve leaflets closer together and providing improved closure during ventricular contraction.

Annuloplasty rings may be rigid, flexible, or have both rigid and flexible segments. Rigid annuloplasty rings have the disadvantage of causing the mitral valve annulus to be rigid and unable to flex in response to the contractions of the ventricle, thus inhibiting the normal movement of the mitral valve that is required for it to function optimally. Flexible annuloplasty rings are frequently made of Dacron® fabric and must be sewn to the annular ring with a line of sutures. Scar tissue formation from the multiple stitches may lead to loss of flexibility and function of the mitral valve. Similarly, combination rings must generally be sutured in place and also cause scar tissue formation and loss of mitral valve flexibility and function.

Another repair procedure involves placing a splint assembly transverse a heart chamber. U.S. Pat. No. 6,723,038 discloses a device for improving mitral valve function that includes placing an elongate member transverse a heart chamber so that each end of the elongate member extends through a wall of the heart. First and second anchoring members are placed external the chamber. The first and second anchoring members are attached to first and second ends of the elongate member to fix the elongate member in a position across the chamber so as to reposition papillary muscles within the chamber. By extending through the walls of the heart, this device risks damage to structures such as the pericardium that lie immediately outside the heart. In addition, multiple separate procedures are required if multiple splints are to be positioned. The splints must each be anchored separately, requiring two openings in the heart walls for each splint positioned.

Therefore, it would be desirable to provide a device, system, and method suitable for treating mitral valve regurgitation that overcome the aforementioned and other disadvantages.

### SUMMARY OF THE INVENTION

One aspect of the present invention is a device for anchoring multiple filaments, comprising an anchor and a tensioner. The anchor has a central aperture, and the tensioner is received within this aperture. The tensioner includes a plurality of openings to receive a plurality of filaments.

Another aspect of the present invention is a system for reducing a compass of a lumen or structure in a mammalian body. The system comprises an anchor having a central aperture, a tensioner having a plurality of openings, and a plurality of filaments, each filament including a retaining member affixed to a distal portion of the filament. The tensioner is receivable within the central aperture of the anchor. A proximal portion of each filament is receivable within a tensioner opening. As used herein, the terms “distal” and “proximal” are with reference to the treating clinician during deployment of the device. “Distal” indicates a portion distant from, or a direction away from, the clinician; and “proximal” indicates a portion near to, or a direction toward, the clinician.

Yet another aspect of the present invention is a method of reducing a compass of a lumen or structure in a mammalian body. An anchor is delivered to a first location proximate the lumen or structure within the mammalian body. Multiple filaments are delivered to a second location across the lumen or structure from the anchor, the filaments positioned spaced apart one from another at the location. The filaments are threaded through the anchor. The filaments are positioned in openings formed in a tensioner and are adjusted within the openings to reduce the compass of the opening or structure in the mammalian body.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is an isometric view of one embodiment of a device for anchoring multiple filaments, in accordance with the present invention, the device shown with the tensioner separate from the anchor;

FIG. 2 is an isometric view of a system for reducing a compass of a lumen or structure in a mammalian body, in accordance with the present invention;

FIG. 3 is an isometric view of an alternative embodiment of a device for anchoring multiple filaments, in accordance with the present invention;

FIG. 4 is a schematic view illustrating placement of the system of FIG. 2 proximate a mitral valve; and

FIG. 5 is a flow diagram of one embodiment of a method of reducing a compass of a lumen or structure in a mammalian body, in accordance with the present invention.

The same reference numbers are used throughout the drawings to refer to the same parts.

### DETAILED DESCRIPTION OF THE PRESENTLY PREFERRED EMBODIMENTS

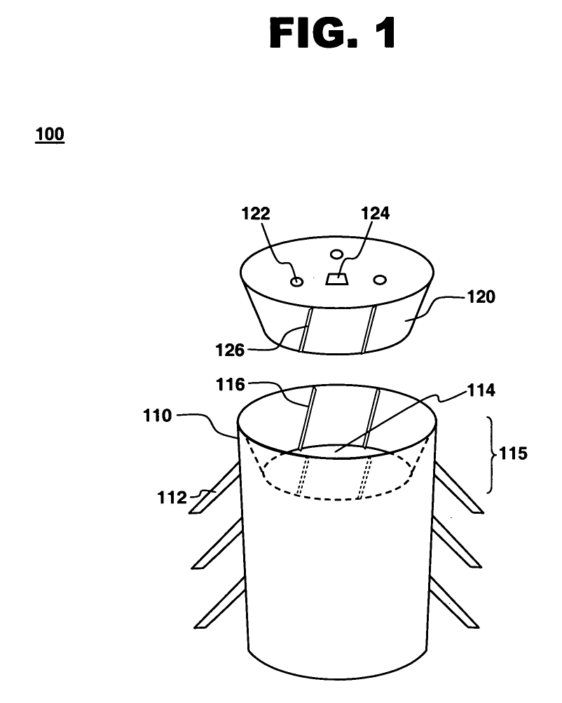

One aspect of the present invention is a device for anchoring multiple filaments. One embodiment of the device, in accordance with the present invention, is illustrated in FIG. 1 at 100. Anchoring device 100 comprises anchor 110 and tensioner 120. Barbs 112 are positioned on the outer surface of anchor 110, and aperture 114 extends through the center of the anchor. Tensioner 120 includes openings 122 to receive a plurality of filaments and coupling structure 124 to releasably couple tensioner 120 with a torquing device.

Anchor 110 comprises one or more biocompatible metallic or polymeric materials. In the present embodiment, anchor 110 is a substantially tubular structure having six barbs 112 positioned on the outer surface of the anchor. The barbs are angled to prevent the anchor from shifting or pulling loose when filaments are received in and tensioned by tensioner 120. The number, arrangement, and shape of the barbs may be varied.

Aperture 114 extends through the center of anchor 110. A proximal portion of central aperture 114 includes tapered seat 115. Tensioner 120, which has a tapered shape complementary to that of tapered seat 115, is received within tapered seat 115 and is prevented from being pulled through anchor 110 by the tapered shape of the seat. In another embodiment, the central aperture and tensioner may not be tapered, and other means, for example recessed shoulders positioned on one or both of the tensioner and the central aperture, may be used to ensure the tensioner cannot be pulled through the anchor.

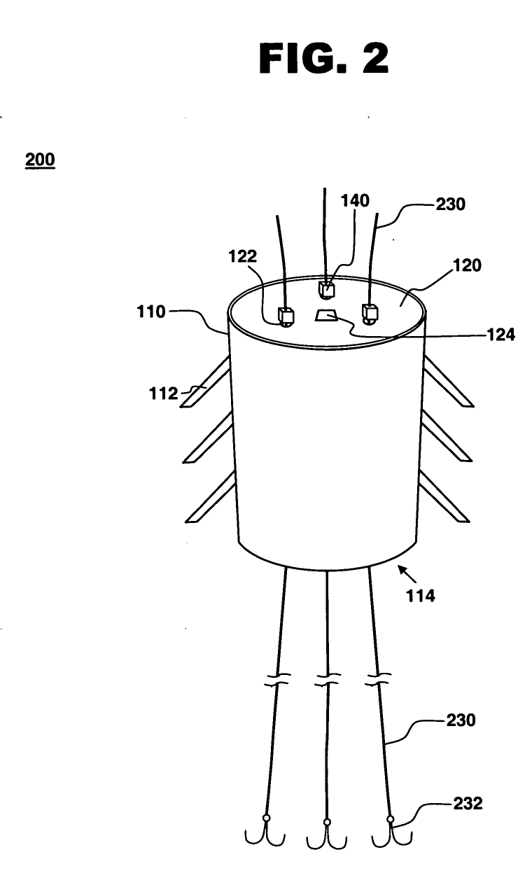

Tensioner 120 includes three openings 122 to receive three filaments. The number of openings may be varied, as well as the number of filaments received within each opening. Once a filament has been threaded into an opening and the tension of the filament adjusted as described below, a locking member may be positioned on the filament adjacent to the opening to retain the filament in the desired position. As shown in FIG. 2, in which like elements share like numbers with FIG. 1, locking member 140 is a device such as is known in the art that may be passed along the filament until the locking member is adjacent to the tensioner opening, at which point the locking member is locked onto the filament. In another embodiment, the locking member may be an integral part of the tensioner opening, for example tensioner opening 122 may allow passage of the filament in just one direction, as is well known in the art. In yet another embodiment, the locking member may attach to both the filament and the opening, thereby locking the filament within the opening.

Tensioner 120 may be fixed within aperture 114 or may be rotatable to wind filaments retained in tensioner openings 122 about each other, reducing the length of the entwined filaments, thereby adjusting the tension exerted by the filaments when anchored at both ends. Where tensioner 120 is rotatable, as in the present embodiment, it is desirable for the tensioner to have rotational freedom of motion in only one of a clockwise or a counterclockwise direction. To prevent the tensioner from rotating in the opposite direction, anchor 110 and tensioner 120 include complementary structures 116 and 126, respectively, that limit the motion of tensioner 120 while the tensioner is positioned within central aperture 114 of anchor 110. In the present example, the structures are capable of ratcheting past each other in only one direction. Only a few structures are shown in FIG. 1; a greater number of structures may be desirable to permit finer positioning. If tensioner 120 is accidentally over-rotated, resulting in greater tension than is desired, the tensioner may be withdrawn from the anchor, disengaging the complementary structures on the tensioner and anchor and allowing the tensioner to rotate in the opposite direction, thereby unwinding the filaments. One skilled in the art will recognize that other structures known in the art may be used to ensure rotational freedom of motion in a single direction. Tensioner 120 may be secured within anchor 110 using an adhesive or mechanical means once the desired tension has been achieved.

Tensioner 120 includes a coupling structure 124 to allow the tensioner to be releasably coupled with a torquing device capable of rotating the tensioner. As shown in FIG. 1, coupling structure 124 is a substantially square opening in tensioner 120 into which a torquing device with a square head may be inserted. The shape of the opening in tensioner 120 and the complementary shape of the torquing device head may be varied. In another embodiment, the coupling structure may extend outward from the tensioner to interface with a complementary receptacle in the torquing device.

Anchoring device 100 is designed to be positioned using a minimally invasive surgical procedure. The tubular shape of anchor 110 makes the structure suitable for implantation into relatively thick, strong tissue such as muscle tissue of the left ventricle adjacent to the mitral valve. When implanted in this cardiac tissue, anchor 110 in combination with tensioner 120 is capable of anchoring multiple filaments to the tissue. It will be obvious to one skilled in the art that device 100 may be implanted into other tissue, including muscle tissue located elsewhere in the body, as well as bone tissue and other types of tissue.

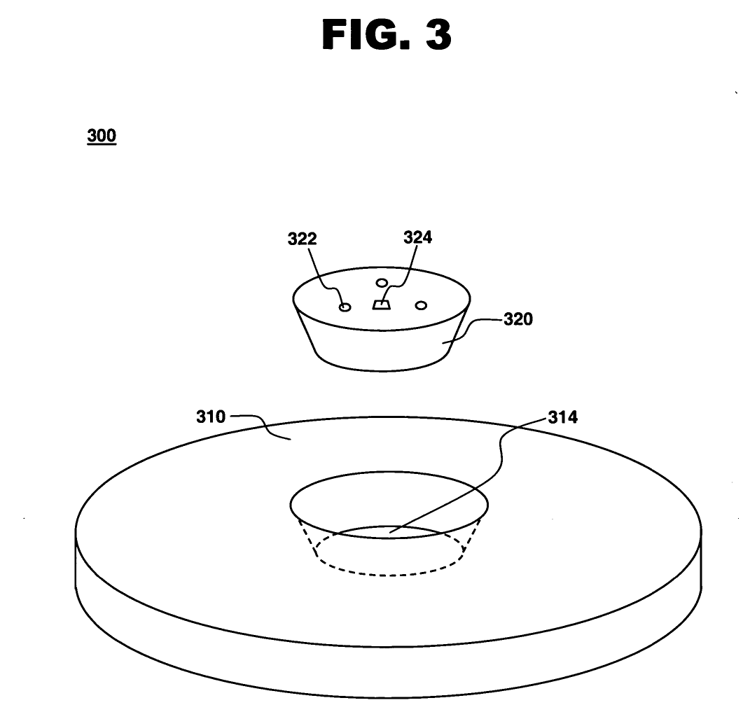

FIG. 3 at 300 shows another embodiment of a device for anchoring multiple filaments, in accordance with the present invention. Anchor 310 is a perforated plate in which the diameter of the perforation, central aperture 314, is substantially smaller than the diameter of the plate. The relatively broad, flat shape of anchor 310 makes device 300 suitable for anchoring multiple filaments to delicate tissue such as cardiac tissue making up the septal wall between the left and right atria of the heart. The relatively large surface area of anchor 310 distributes stress applied to the anchor by the filaments over a similarly large area of the septal wall. One skilled in the art will appreciate that anchoring device 300 may anchor multiple filaments to tissue other than cardiac tissue.

Another aspect of the present invention is a system for reducing a compass of a lumen or structure in a mammalian body. One embodiment of the system, in accordance with the present invention, is illustrated in FIG. 2 at 200. System 200 includes the anchoring device illustrated in FIG. 1, comprising anchor 110 and tensioner 120. System 200 further includes multiple filaments 230. The system is described below in the context of radially contracting a mitral valve annulus to effect a mitral valve repair. However, it will be apparent to one skilled in the art that a system in accordance with the present invention may be used to reduce the compass of other openings and structures within the body.

As described more fully above, anchor 110 is a tubular structure having multiple barbs 112 positioned on an outer surface of the anchor. Anchor 110 includes central aperture 114, within which tensioner 120 is receivable. Tensioner 120 includes multiple openings 122, within which proximal portions of filaments 230 are receivable.

Filaments 230 may be nitinol wires, suture threads, or other biocompatible filaments known in the art. Retaining members 232 are affixed to distal portions of filaments 230. In the present embodiment, each retaining member is an expandable nitinol clip designed to be deployed within cardiac tissue, thereby attaching the distal end of each filament to the tissue. In another embodiment, the retaining members may be other structures known in the art that are suitable for attaching the filaments to tissue within a mammalian body.

As shown in FIG. 2, tensioner 120 includes three openings 122, with one filament 230 received within each opening. The number of openings may be varied, as well as the number of filaments received within each opening. A locking member 140 is positioned adjacent to each tensioner opening 122 to retain a filament 230 within the opening. Locking members 140 may be devices such as those shown in FIG. 2 that are independent from tensioner 120, or the locking members may be integrated into the openings.

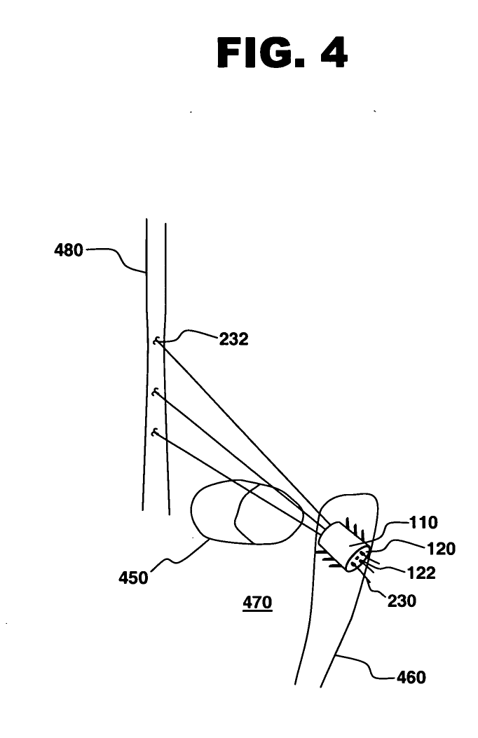

System 200 may be used to reduce or eliminate mitral valve regurgitation by radially contracting the mitral valve annulus. This may be accomplished as illustrated in FIG. 4. Anchor 110 is implanted adjacent to mitral valve 450 within muscle tissue comprising free wall 460 of left atrium 470. Filaments 230 are threaded through the central aperture of anchor 110 and extend to atrial septal wall 480, where retaining members 232 attach the distal ends of the filaments to the septal wall. Proximal portions of the filaments are threaded through openings 122 in tensioner 120, which is then positioned within the central aperture of anchor 110. Locking members retain the filaments within the openings, thereby anchoring the filaments to anchor 110 and the muscle tissue within which the anchor is implanted. One skilled in the art will appreciate that system 200 may also be positioned across the left ventricle, rather than the left atrium, to effect a mitral valve repair.

When properly adjusted, the filaments exert tension across the mitral valve, radially contracting the mitral valve annulus to reduce or eliminate mitral valve regurgitation. The tension of filaments 230 is adjusted first by drawing the filaments proximally through openings 122 and locking the filaments in place using the locking members. Once a filament has been locked to the tensioner, it may be cut and excess length removed from the body. If further adjustment is needed, tensioner 120 may be rotated within the central aperture of anchor 110 to twist the filaments together distal to the tensioner, shortening the length of the entwined filaments. This draws retaining members 232 toward anchor 110, thereby reducing the radial dimension of the mitral valve annulus.

As described above, anchor 110 and tensioner 120 include complementary structures that allow the tensioner to rotate in only a clockwise or a counterclockwise direction, preventing the filaments from unwinding once the proper tension has been achieved. If tensioner 120 is accidentally over-rotated, resulting in greater tension than is desired, the tensioner may be withdrawn from anchor aperture 114. This disengages the complementary structures on the tensioner and anchor and allows the tensioner to rotate in the opposite direction to unwind the filaments. Tensioner 120 may be secured within anchor 110 using an adhesive or mechanical means once the proper tension has been achieved.

One skilled in the art will appreciate that the anchor may take other forms. For example, the anchor may be a perforated plate such as is illustrated at 310 in FIG. 3. To reduce the radial dimension of a mitral valve annulus using an anchor having this shape, the filament retaining members, rather than the anchor, are embedded in free wall muscle tissue adjacent to the mitral valve. Anchor 310 is positioned resting against either the right atrial or right ventricular surface of the corresponding septal wall. The filaments pass through the septal wall via aperture 314 and tensioner openings 322 and are retained within openings 322. The tension of the filaments may be adjusted both by adjusting the filaments within the openings and by rotating tensioner 320 to wind the filaments about one another, thereby shortening the length of the entwined filaments.

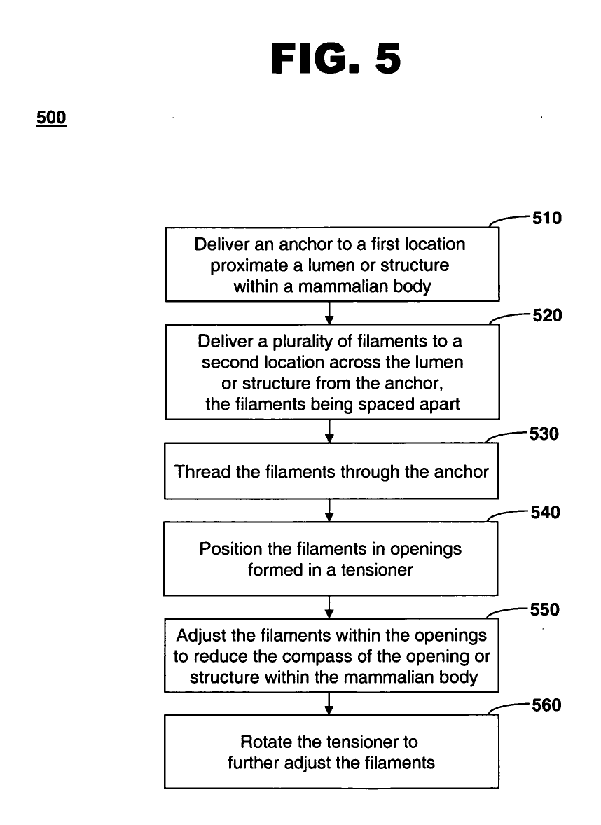

Another aspect of the present invention is a method of reducing a compass of a lumen or structure in a mammalian body. FIG. 5 shows a flow diagram of one embodiment of the method in accordance with the present invention. The described method is intended to reduce the diameter of a mitral valve annulus to effect a mitral valve repair.

An anchor is delivered to a first location proximate the lumen or structure within the mammalian body (Block 510). In the present embodiment, the anchor is delivered into free wall muscle tissue adjacent to the mitral valve. This is accomplished by tracking to the target location with a wire and following with a guide. The anchor is delivered over the wire and released.

A plurality of filaments are delivered to a second location across the lumen or structure from the anchor (Block 520), the second location being either the left atrial side or the left ventricular side of the corresponding septal wall. The filaments are delivered one at a time to spaced apart positions on the septal wall and are attached to the wall using retaining members positioned on the distal ends of the filaments.

The filaments are threaded through the anchor (Block 530). In the present embodiment, a delivery system used to implant each filament within the septal wall is inserted through the central aperture of the anchor and tracks to the target location on the septal wall. The filaments are attached to the wall, and the delivery system is withdrawn back through the anchor aperture, thereby threading the filaments through the anchor. In another embodiment, the filaments may be delivered before the anchor, in which case the proximal ends of the filaments would have to be threaded through the central aperture of the anchor prior to delivery of the anchor.

The filaments are positioned in openings formed in a tensioner (Block 540). At this point in the method, the tensioner is outside of the body, separate from the anchor. The proximal ends of the filaments, which extend outside the body, are individually threaded through the tensioner openings, one filament in each opening. The tensioner is then delivered over the filaments until it is positioned in the central aperture of the anchor.

The filaments are adjusted within the openings to reduce the compass of the lumen or structure in the mammalian body (Block 550). Each filament is drawn proximally through the tensioner opening until the filament is taut and exerting some tension on the mitral valve annulus. Once adjusted, the filaments are retained within the openings using either a locking member integrated into the opening or a locking member that is passed along the filament until the locking member is adjacent to the tensioner opening, at which point the locking member is locked onto the filament. Once a filament has been locked to the tensioner, it may be cut and excess length removed from the body.

Simply pulling the filaments taut within the tensioner openings may provide sufficient tension to reduce the diameter of the mitral valve annulus and effect a mitral valve repair. Where additional tension is required to minimize or eliminate mitral valve regurgitation, the tensioner is rotated to further adjust the filaments (Block 560). Rotating the tensioner twists the filaments together, thereby shortening the length of the entwisted filaments and further reducing the diameter of the mitral valve annulus. The tensioner includes a coupling structure that allows the tensioner to be releasably coupled to and rotated by a torquing device that is inserted into the body to rotate the tensioner and then withdrawn once rotation has been completed. Functioning of the valve may be monitored using Doppler techniques during tensioning of the filaments to provide optimal valve repair. Once the desired tension has been achieved, the tensioner is secured mechanically or with an adhesive to prevent the tensioner from rotating in the reverse direction.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A device for anchoring multiple filaments, comprising:
an anchor having a central aperture formed therein; and
a tensioner receivable within the central aperture of the anchor, the tensioner including a plurality of openings to receive a plurality of filaments.

2. The device of claim 1 wherein the tensioner is rotatable within the central aperture of the anchor.

3. The device of claim 2 wherein the anchor and the tensioner include complementary structures that prevent the tensioner from rotating in one of a clockwise or a counterclockwise direction.

4. The device of claim 2 wherein the tensioner includes a coupling structure to releasably couple the tensioner with a torquing device.

5. The device of claim 1 wherein the anchor includes a plurality of barbs positioned on an outer surface of the anchor.

6. The device of claim 1 wherein a locking member positioned adjacent to a tensioner opening retains a filament within the opening.

7. The device of claim 1 wherein the anchor comprises a perforated plate.

8. The device of claim 1 wherein the anchor comprises a tubular member.

9. The device of claim 1 wherein the device anchors multiple filaments to cardiac tissue.

10. A system for reducing a compass of a lumen or structure in a mammalian body, comprising:
an anchor having a central aperture formed therein;
a tensioner receivable within the central aperture of the anchor, the tensioner including a plurality of openings; and
a plurality of filaments, each filament including a retaining member affixed to a distal portion of the filament, wherein a proximal portion of each filament is receivable within a tensioner opening.

11. The system of claim 10 further comprising:
a locking member positioned adjacent to each tensioner opening, wherein each locking member retains a filament within the opening.

12. The system of claim 10 wherein the tensioner is rotatable within the central aperture of the anchor.

13. The system of claim 11 wherein the anchor and the tensioner include complementary structures that prevent the tensioner from rotating in one of a clockwise or a counterclockwise direction.

14. The system of claim 12 wherein the anchor includes a plurality of barbs positioned on an outer surface of the anchor.

15. The system of claim 11 wherein rotating the tensioner reduces the radial dimension of a mitral valve annulus.

16. A method of reducing a compass of a lumen or structure in a mammalian body, the method comprising:
delivering an anchor to a first location proximate a lumen or structure within a mammalian body;
delivering a plurality of filaments to a second location across the lumen or structure from the anchor, the filaments being positioned spaced apart one from another;
threading the filaments through the anchor;
positioning the filaments in openings formed in a tensioner; and
adjusting the filaments within the openings to reduce a compass of the lumen or structure in the mammalian body.

17. The method of claim 16 further comprising:
rotating the tensioner to adjust the filaments.

18. The method of claim 16 wherein delivering an anchor to a first location proximate target tissue within the mammalian body comprises delivering the anchor into muscle tissue of the left ventricle wall adjacent to the mitral valve.

19. The method of claim 18 wherein delivering a plurality of filaments to a second location proximate the target tissue comprises delivering the filaments into an atrial septal wall adjacent to the mitral valve.

20. The method of claim 16 wherein adjusting the positioned filaments within the tensioner reduces a diameter of a mitral valve annulus to effect a mitral valve repair.
